(19) United States
(12) Patent Application Publication
(10) Pub. No.: US 2006/0179583 A1
(43) Pub. Date: Aug. 17, 2006

(54) Processes

(75) Inventors: Hal Ambuter, Montvale, NJ (US); James William Cavanagh, Montvale, NJ (US)

Correspondence Address: NORRIS, MCLAUGHLIN & MARCUS, 875 THIRD AVE, 18TH FLOOR, NEW YORK, NY, 10022, US

(73) Assignee: Reckitt Benckiser Inc., Parsippany, NJ (US)

(21) Appl. No.: 10/565,924

(22) PCT Filed: Jul. 30, 2004
(86) PCT No.: PCT/GB04/03296

§ 371 Date: Apr. 24, 2006

(30) Foreign Application Priority Data
Jul. 30, 2003 (US) 10/631949

(51) Int. Cl. B08B 7/04; B08B 9/20; A01H 5/02; D06F 35/00

(52) U.S. Cl. 8/158; 8/148; 134/25.2; 134/18

(57) ABSTRACT

The present invention provides for a method of dosing treating agents into a washing machine in particular sequences so that only one treating agent is dosed at one time. This avoids interaction of incompatible treating agents as well as provide for better cleaning as now one treating agent can perform without interference from other treating agents typically found in washing machines.

This invention relates to the process of using multiple detergent compositions, rinse aids, and other additives within one complete wash cycle of an automatic washing machine. The various cleaning compositions may be dosed into the machine at varying quantities, times, sequences, and for varying durations during a washing machine cycle. The use of multiple cleaning compositions allows for increased and optimized cleaning performance.

Current conventional systems used in automatic dishwashers only dose one detergent composition per wash cycle with the optional addition of a rinse agent composition at the very end of the washing machine cycle. The detergent compositions are primarily either enzymatic based or incorporate a hypohalite oxidative bleach (e.g. sodium hypochlorite, sodium dichloroisocyanurate, etc.).

Enzymatic detergents provide excellent cleaning on enzyme sensitive soils (primarily protein and starch based) but fail to provide performance on hard to remove stains, such as coffee, tea, and tomato stains.

Hypohalite based (for example, chlorine bleach based) detergents provide excellent cleaning on the hard to remove stains but fail to provide performance on the enzyme sensitive soils.

Because enzymes and hypohalite oxidizing bleaches are incompatible within the same formula matrix, the consumer must make a trade-off decision on performance and use one detergent composition or the other. This presents an obvious dilemma to the consumer—whether to get good cleaning on an enzymatic sensitive stain to the detriment of a hard to remove stain or vice versa.

The use of multiple detergent compositions within one washing machine cycle would mitigate this trade-off decision and provide optimal performance across the range of stains and soils normally encountered in an automatic dishwasher. However, given the incompatibility of enzyme based detergents and hypohalite detergents, the detergent compositions must be kept separate and dosed at different times so that the performance of each detergent is not affected by the presence of the other detergent.

Thus, an object of the present invention is to provide a multistage automatic dishwashing machine where different detergents and other additives can be dosed into the machine at different times during the machine cycle so that a particular dosed detergent or additive can perform its function without interference or deleterious effects from other detergents or additives. Once the particular dosed detergent or additive has performed its function, a further detergent or additive can be dosed into the machine to perform yet another function.

Another object of the present invention is to provide for a cartridge that contains at least two chambers with each chamber containing one discrete detergent or additive. The individual chambers can be charged with a variety of detergents and additives that can be used to treat the contents of the washing machine as well as the interior of the washing machine itself.

Typically dishwashing machines contain a suitable slot or receiving port of appropriate size to accommodate the cartridge. Additionally, the machine usually contains electrical and/or mechanical circuitry, sensors and/or actuators and/or logic controllers, which permit the user to operate the machine by selecting a particular cycle (or alternatively by selecting a series of segments to define a particular cycle). The use of a controller, the construction and design of which is well known to those skilled in the art, allows the selected cycle/segments to send an appropriate signal to the cartridge such that at the appropriate segment of the cycle, (as either preprogrammed by the machine manufacturer or programmed by the user), the appropriate chamber in the cartridge is activated and the material therein is dosed.

Alternatively the dosing may be based upon feedback from a sensor within the machine that determines a feature of the load such as the size thereof and/or the amount of soil therein. In this way a desired chamber in the cartridge may then be activated. At the same time, one or more other chamber(s) may be “locked out”, unable to dose its (their) material into the machine.

In addition or as an alternative the sensors within the machine may be used to detect the type or quality of load or water hardness at the appropriate time. Generally, but not always, this occurs at the beginning of the cycle. Detecting the number of items and/or soil load then permits the machine to devise a series of segments that would dose appropriate treating agents in the appropriate segment.

In certain instances, it may be desirable to dose an enzymatic detergent first, then followed by a hypohalite detergent and then finally with a rinse aid. In other instances, it may be desirable to dose a hypohalite detergent first, then followed by an enzymatic detergent and then finally with a rinse aid. In further instances, it may be desirable to dose an enzymatic detergent first, then followed by a rinse aid; then followed by a hypohalite detergent and then finally with a rinse aid. In still further instances, it may be desirable to dose a hypohalite detergent first, then followed by a rinse aid; then followed by an enzymatic detergent and then finally with a rinse aid. In even still further instances, it may be desirable to first dose water treatment agents (for example, builders, water softeners, chelaters, etc and the like) and then follow with either an enzymatic detergent or hypohalite detergent, then either a hypohalite detergent or enzymatic detergent, and then a rinse aid. Even further instances may include a segment where a dose of anti-lime scale agent is dosed prior to the final rinse aid segment. In even further instances, it may be desirable to dose an additive (for example, a rinse aid) at the same time as the hypohalite detergent or enzymatic detergent. Those in the art will appreciate that there are numerous other segment combinations which can be envisioned, all of which are within the scope of the present invention.

Depending upon the treating agent to be dosed into the machine, the dosing of the detergent may take place prior to the final rinse segment or zone, preferably prior to the first wash segment or zone.

A further object for the present invention is to provide a method of dispensing a plurality of treating compositions into a multi-stage automatic washing machine, the method comprising operating a cartridge in the machine, the cartridge comprising at least two chambers, each chamber containing a treating composition, wherein the chambers are activated in a manner such that only one chamber is activated and one treating composition is does during each stage of the dishwashing cycle.

Most preferably the automatic washing machine is an automatic dishwashing machine.

Optionally a plurality of cartridges may be provided within the automatic dishwashing machine, wherein each cartridge has a plurality of chambers for holding/dosing a treating composition.

Most preferably the chambers of the cartridge contain at least two different treating compositions. Optionally each treating composition differs from each other treating composition.

The treating composition may comprise a single treating agent or alternatively may comprise a plurality of treating agents.

The types of treating agents which can be placed individually into the separate chambers include enzymatic detergents, hypohalite/peroxygen detergents, water treatment agents, rinse aids, anti-lime scale removers, sanitizers, perfumes, and surface repair agents.

A typical dishwashing cycle consists of a pre-rinse segment, a wash segment, two more rinse segments, and finally, a dry segment. Some dish washing machines may have an additional segment such as treating segments (for example, a water treatment segment or an anti-lime scale segments). A timing device within the dishwasher is responsible for precisely controlling all of the electrical circuits and activating the components associated with each segment.

Preferably the cartridge chamber that is activated in the pre-rinse segment contains an enzymatic detergent.

Preferably the cartridge chamber that is activated in the wash segment contains a hypohalite/peroxygen detergent.

Preferably the cartridge chamber that is activated in the rinse segment contains a rinse agent.

Preferably the cartridge chamber that is activated in the treatment segment contains an anti-lime agent or a water treatment.

To clearly illustrate this concept the operation of the cartridge in accordance with the method of the present invention in a typical dishwashing machine may be as follows.

For use with a typical multistage dishwashing machine the cartridge comprises four chambers, one for each of the cycles outlined above. Each cartridge chamber, independently of the other cartridge chambers may be filled, partially filled or empty. The filling of each cartridge may be dependent upon the nature of the dishwasher machine cycle, e.g. whether or not a particular segment is present in said cycle. Alternatively the user may exert some influence as to the needs of the items to be washed and the amount of treating composition added to each chamber.

The cartridges may also be sold commercially, wherein the treating agents have been added as necessary to each cartridge chamber.

Usually chamber one (for activation in a pre-rinse segment) contains an enzymatic detergent, chamber two (for activation in a wash-segment) contains a hypohalite detergent, chamber three (for activation in a rinse segment) contains a rinse aid, and chamber four (for activation in a treatment-segment) contains a water treatment agent. Chambers one, two, three, and four are activated during the machine dishwasher cycle in a sequential manner to dose their respective contents (if present) into the machine during a predetermined segment such that only one chamber is activated and the material therein is dosed into the machine during said segment no other chamber is activated and no other material is dosed into the machine until the prior stage has been completed.

Typical preprogrammed cycles found in automatic dishwashing machines and cycles include HEAVY and CHINA CRYSTAL. Within these and other automatic dishwasher cycles, (which can, for example, be selected by the user) is an array of options. Examples of options include DELAY START, AIR DRY, LOW ENERGY RINSE, HIGH TEMP WASH, and CANCEL DRAIN.

Each cycle can have its own treating agent dispense requirements, for example, for a HEAVY cycle, it may be preferred or necessary to first dose a rinse agent then followed by an enzymatic detergent and then the hypohalite detergent (or vice versa) and then finally an anti-lime scale agent.

In another example, for a CHINA CRYSTAL cycle, it may be preferred or necessary to first dose a rinse agent, then an enzymatic detergent (or hypohalite detergent), then the rinse agent, then a hypohalite detergent (or enzymatic detergent), and then finally again a rinse agent.

For a typical automatic dishwasher machine, once the machine is loaded with articles to be cleaned and/or treated, generally the following events occur when the door of the washing machine is closed and the user has selected a particular cycle (either preprogrammed or programmed).

(1) Latching the door activates the timer and other controls. The user selects a cycle by pressing a button and/or turning a dial on the front panel of the dishwasher.

(2) The timer opens a water-inlet valve and when the water reaches the appropriate level in the dishwasher tub, the water-inlet valve closes. The timer advances to activate a motor-driven pump, which sends water through the pump housing and into the spray arms and tower at a powerful rate, causing the spray arms to rotate and spray water over the dishes.

(3) As the water becomes soiled with food particles, the water circulates through a filtration system which eliminates food particles from the water.

(4) At the end of the rinse segment, the timer signals the machine to empty the water into the home's drain system. If a cycle requires another rinse segment, the timer activates the machine to refill, rinse and drain before going into the main wash segment.

(5) For the main wash segment, the timer signals the detergent dispenser to open and empty its contents into the water-filled tub.

(6) The hot water and detergent are pumped throughout the machine to break down and loosen soil on dishes and utensils. The timer then directs the pump to drain the tub and refill with clean, hot water for final rinse segments.

(7) Once the final rinse segments are completed, the automatic drying period begins.

As can be appreciated, at certain points within the above cycles, the treating agents discussed herein can be dosed into the washing machine to perform rinsing, cleaning, disinfecting, water treating, and other tasks for which the treating agents are designed.

For example, during segment (2), a water treatment agent could be dosed into the washing machine to address any water hardness issues. Of course this will vary depending upon the water quality of the individual user. Thereafter, a rinse agent could also be dosed.

For segment (5), an enzymatic detergent could be dosed first into the washing machine and allowed to work. Then a segment (5A) could be envisioned where there is a short rinse and then segment (5B) would then dose a hypohalite detergent. Then segment (6) would then follow.

As mentioned above, there can be a variety of different segments which can be placed in a variety of, sequences to define a cycle. The various cycles can be preprogrammed by the washing machine manufacturer or could be programmed by the user. Also envisioned are sensors within the washing machine that could sense the article load and the soil load. In so doing, the amount of treating agent to be dosed could be changed to meet the load requirements.

In practice, the washing machine user will load the washing machine with articles to be cleaned. After selecting a preprogrammed cycle or selecting segments which form a cycle, the washing machine is turned on.

Water hardness sensors can be placed, for example, in the water inlet pipe into the washing machine or in the bottom of the washing machine cavity. The water hardness sensor could be an ion selective electrode or detectors which can measure the amount of calcium and/or magnesium in the water. The sensor can be preset such that depending upon the hardness of the water, an appropriate amount of water treating agent can be added. Water hardness is classified by the U.S. Department of Interior and the Water Quality Association and can range from soft water (0-17 mg/l or ppm of hardness) to moderately hard water (60-120 mg/l or ppm of hardness) to hard water (120-180 mg/l or ppm of hardness) to very hard water (>180 mg/l or ppm of hardness). The amount of water treatment agent needed to be added to adjust the incoming water to an appropriate water hardness can be programmed into the sensor. Additionally, various types of water treatment agents are available and the sensor can be programmed to identify the water treatment agents in the cartridge through manufacturer's sensors identifying the agents which are placed on a cartridge.

Once the water hardness has been adjusted to an appropriate level, infrared and/or ultra violet sensors which are placed within the washing machine can do a survey of the load to determine the type and quantity of load. For example, the IR and/or UV sensors could send out signals to survey the load. Both enzyme sensitive and hard to remove stains, as discussed above, could be detected. If the majority of the stains were detected to be hard to remove stains, for example, red containing stains which could be indicative of a tomato based stain—identified above as preferably treated by the use of a hypohalite detergent. If detected, then a logic switch connected to the sensor would then send a signal to the chamber containing the hypohalite to be dispensed and thus a first wash segment could be commenced. Thereafter, once this wash segment was complete, the water in the cavity could be discharged, new water loaded, again check for water hardness, and then the enzymatic detergent could be charged into the machine and the second wash segment could commence. Once this wash segment was complete, the water in the cavity could be removed and the rinse segment(s) could commence.

Those in the art will appreciate that if the IR and/or UV sensors detected more protein type stains (for example, egg), then the first wash segment would be conducted using an amount of enzymatic detergent dosed into the cavity. The second wash segment would then be conducted using the hypohalite detergent.

The invention is further illustrated with reference to the following non-limiting Examples.

### EXAMPLES

The performance of dosing different detergents at different times within a washing machine was investigated.

Two performance tests were run to illustrate the performance differences between the use of a sole detergent and a multiple detergent system. The tests compared the performance of the following detergent systems:

Ex. 1 enzymatic detergent alone in the pre-wash cup and in the main wash cup

Ex. 2 hypohalite detergent alone in the pre-wash cup and in the main wash cup

Ex. 3 enzymatic detergent in the pre-wash cup and hypohalite detergent in the main wash cup

Ex. 4 hypohalite detergent in the main wash cup and enzymatic detergent in the main wash cup

The first test was a cleaning test that measures the performance of a detergent system at removing a wide variety of different types of soils and stains. The test used the European standard IKW (Industrial Associate for Personal Hygienic Products and Detergents) cleaning test in commercially available US machines against four different soil/stain classes: bleachable, dried/starch containing, dried/protein and, persistent/burnt containing. The soil/stains tested are listed in Table 1. The detergents tested and other testing parameters and equipment are set forth in Table A. Each soil/stain was visually rated after the wash performance using a scoring system of 1 to 10; 10 indicating 100% removal and 1 indicating 0% removal. The results are found in Table 1.

Hypohalite Detergent (HD)

| Component | Wt % |
|---|---|
| De-hardened water | 61.3 |
| Monopropylene glycol | 1.9 |
| Isothiazolinone | 0.1 |
| Polyacrylic acid | 1.25 |
| Trisodium citrate | 32.8 |
| Calcium chloride | 0.5 |
| Sodium hydroxide(50%) | 0.23 |
| Nonionic EO/PO LF 500 | 0.2 |
| Available Chlorine | 1.0 |
| (present as sodium hypochlorite) | |

Enzymatic Detergent (HE)

| Component | Wt % |
|---|---|
| Citric Acid | 10.0 |
| Na2CO3/K2CO3 | 1.9 |
| Dispersant | 4 |
| HEDP | 1.5 |
| Benzoyl peroxide | 1.5 |
| BHT | 1.5 |
| Boric Acid | 4.0 |
| Sorbitol | 6.0 |
| Savinase | 0.5 |
| Maxamyl/Termamyl | 0.3 |
| Water | To 100 |

TABLE A

| | |
|---|---|
| Hypohalite Detergent | HD |
| Enzymatic Detergent | ED |
| Water hardness | 9° GH |
| Water temperature | 55° C. |
| Machine: | G.E. Potscrubber |
| Program: | Normal/Heated Dry |

TABLE 1

| | Ex. 1 | Ex. 2 | Ex. 3 | Ex. 4 |
|---|---|---|---|---|
| Pre-wash Detergent Cup 45 gram dosage | HD | ED | HD | ED |
| Main wash Detergent Cup 60 gram dosage | ED | HD | HD | ED |
| Bleachable Stains | | | | |
| TEA INTENSIVE | 6.7 | 9.8 | 9.9 | 4.0 |
| Starch, dried-on | | | | |
| OAT FLAKES | 8.9 | 8.3 | 5.4 | 9.0 |
| STARCH MIX | 9.8 | 9.0 | 7.0 | 10.0 |
| Protein, dried-on | | | | |
| MINCED MEAT | 10.0 | 1.8 | 3.9* | 9.8 |
| EGG YOLK | 6.1 | 2.0 | 0.8 | 6.5 |
| EGG YOLK/MILK | 7.5 | 2.4 | 1.2 | 7.9 |
| Burnt-on Stains | | | | |
| MILK | 7.4 | 7.5 | 7.9 | 7.3 |
| Average | 8.06 | 5.83 | 5.16 | 7.78 |

As can be seen from the data in Table 1, a combination of an enzymatic and hypohalite detergents provided superior cleaning performance results versus using either one of the detergents alone.

The second test measured the spotting and filming performance of the detergent systems evaluated above. This test used the standard ASTM method D3556-85 (Reapproved 1989) “Standard Test Method for Deposition on Glassware During Mechanical Dishwashing” to evaluate spotting and filming in commercially available US machines The detergents tested and other testing parameters and equipment are set forth in Table B. Glasses were measured for spotting and filming after 4 consecutive washes. Spotting and filming were graded separately for each glass on a scale from 1 to 5, 1 indicating no spotting or filming and 5 indicating heavy level of spotting and filming. The results are found in Table 2.

TABLE B

| | |
|---|---|
| Hypohalite Detergent | HD |
| Enzymatic Detergent | ED |
| Test Method | ASTM D3556-85 Spotting & Filming |
| | Test |
| Water hardness | 150 ppm calcium carbonate |
| Water temperature | 120° F. (˜49° C.) |
| Machine | G.E. Profile |
| Program | Normal/Heated Dry |

TABLE 2

| | Ex.5 | Ex. 6 | Ex. 7 | Ex. 8 |
|---|---|---|---|---|
| Pre-wash Detergent Cup 45 gram | HD | ED | HD | ED |
| dosage | | | | |
| Main wash Detergent Cup 60 gram | ED | HD | HD | ED |
| dosage | | | | |
| Spotting | 1.64 | 1.71 | 1.86 | 1.64 |
| Filming | 1.75⁷⁸ | 1.48⁷⁸ | 2.15 | 2.06 |

The superscript letter above the individual score indicates a significant difference versus the product indicated. As an example the score for filming in Ex. 5 is indicated as 1.75⁷⁸. This means that it is significantly better in filming than Ex. 7 and Ex. 8.

The data in Table 2 show that a combination of an enzymatic and hypohalite detergent system produce superior cleaning performance results versus using either one of the detergents alone.

The above data highlight the advantages of a multiple cleaning composition system when used in current, commercially available machines, which have preset detergent dosage amounts and preset wash cycle times. Further performance advantages can be achieved if these restrictions are removed through redesign of the dishwasher.

The cleaning system could be either

1. placed into the existing dishwasher cups

2. placed into a bulk storage reservoir permanently attached to the dishwasher

3. placed into a disposable or reusable cartridge

Additionally, further cleaning compositions can be added to this detergent system for even higher levels of performance apart from those mentioned above. For example:
1. multiple dosing points in the wash segment of rinse agent—currently limited to a dosage of 1-3 ml and only at the very end of the wash segment.
2. The addition of an acidic based composition at the end of the wash segment to remove any limescale or soap scum deposits from either the dishes/plates/glasses or the machine itself.
3. The addition of a cationic disinfectant composition at the end of the wash segment.

1. A method of dispensing a plurality of treating compositions into a multistage automatic washing machine comprising operating a cartridge in the machine, the cartridge comprising at least two chambers, each chamber containing a treating composition, wherein the chambers are activated in a manner such that only one chamber is activated and one treating composition, is dosed during each stage of the washing cycle.

2. The method of claim 1 wherein a plurality of cartridges are provided within the automatic washing machine.

3.-14. (canceled)

15. The method of claim 1, wherein the chambers of the cartridge contain a plurality of treating compositions.

16. The method of claim 2, wherein the chambers of the cartridge contain a plurality of treating compositions.

17. The method of claim 15, wherein each treating composition differs from the other treating compositions.

18. The method of claim 1 wherein the cartridge comprises 4 chambers.

19. The method of claim 1, wherein the cartridge comprises a chamber suitable for activation in a pre-rinse segment, which contains an enzymatic detergent treating composition.

20. The method of claim 1, wherein the cartridge comprises a chamber suitable for activation in a wash segment, which contains a hypohalite/peroxygen detergent treating composition.

21. The method of claim 1, wherein the cartridge comprises a chamber suitable for activation in a rinse segment, which contains a rinse agent treating composition.

22. The method of claim 1, wherein the cartridge comprises a chamber suitable for activation in a treatment segment, which contains an anti-lime agent or a water treatment agent treating composition.

23. The method of claim 1, wherein in operation the cartridge interacts with a sensor within the automatic washing machine, the sensor sensing a parameter of the automatic washing machine wash liquor and conveying the parameter back to the cartridge, influencing the operation of a cartridge chamber.

24. The method of claim 23, wherein the sensor senses the hardness of the water in the automatic washing machine wash liquor.

25. The method of claim 23, wherein the sensor senses the soil loading of the water in the automatic dishwasher machine wash liquor.

26. The method according to claim 23, wherein the sensor senses the amount to which the automatic washing machine has been loaded with house ware to be washed.

27. The method of claim 2, wherein in operation the cartridge interacts with a sensor within the automatic washing machine, the sensor sensing a parameter of the automatic washing machine wash liquor and conveying the parameter back to the cartridge, influencing the operation of a cartridge chamber.

28. The method of claim 27, wherein the sensor senses the hardness of the water in the automatic washing machine wash liquor.

29. The method of claim 27, wherein the sensor senses the soil loading of the water in the automatic dishwasher machine wash liquor.

30. The method according to claim 27, wherein the sensor senses the amount to which the automatic washing machine has been loaded with house ware to be washed.
